(19) United States
(12) Patent Application Publication
(10) Pub. No.: US 2022/0204522 A1
(43) Pub. Date: Jun. 30, 2022

(54) PROCESS FOR SEPARATING AND PURIFYING ARTEMISININ

(71) Applicant: HUNAN HEALTHWARE BIOTECH LTD., Hunan (CN)

(72) Inventors: Mei Zhang, Jiangsu (CN); Jinzhao Zhao, Jiangsu (CN); Jinquan Zhao, Hunan (CN)

(73) Assignee: HUNAN HEALTHWARE BIOTECH LTD., Hunan (CN)

(21) Appl. No.: 17/610,442

(22) PCT Filed: Feb. 24, 2020
(86) PCT No.: PCT/CN2020/076403

§ 371 Date: Nov. 11, 2021

(30) Foreign Application Priority Data
Apr. 11, 2019 (CN) 201910291762.4

(51) Int. Cl. C07D 493/18

(52) U.S. Cl. CPC C07D 493/18

(57) ABSTRACT

The present invention belongs to a process for separating and purifying artemisinin comprising S1. concentrating and pressure filtering an artemisinin extraction liquid to obtain a crude artemisinin crystal 1 and a mother liquor 1, and concentrating the mother liquor 1 to obtain an artemisinin extract 1; S2. adding the artemisinin extract 1 to an aqueous alcohol solution to obtain an aqueous alcohol extraction liquid 1 and an artemisinin extract 2; S3. subjecting the artemisinin extract 2 to S2 so as to obtain an aqueous alcohol extraction liquid 2 and an artemisinin extract 3; S4. combining the aqueous alcohol extraction liquids 1 and 2 to obtain a crude artemisinin crystal 2 and a mother liquor 2; and S5. combining the crude artemisinin crystals 1 and 2, then dissolving same in an alcohol solvent and filtering same, heating the filtrate and then concentrating same to obtain a fine artemisinin product.

### TECHNICAL FIELD

The present invention belongs to the technical field of separation and purification of artemisinin, and specifically relates to a process for separating and purifying artemisinin.

### DESCRIPTION OF RELATED ART

Artemisinin is a sesquiterpene lactone compound with a peroxo bridge. It was extracted and isolated from the leaves of the Artemisia annua Linn. (a composite plant) by Chinese pharmaceutical workers in 1971. Plant extraction, biosynthesis, chemical synthesis and plant tissue culture are the main artemisinin preparation processes. Since various components of Chinese herbal medicine have different solubility in different solvents, the effective components can be extracted, which can be used as a basis for the direct extraction of artemisinin from Artemisia annua. How to extract artemisinin more efficiently and reduce the energy loss in the extraction process while ensuring the purity of the product is a technical problem that needs to be solved in this field.

There have been many reports on the field of extraction, separation and purification methods of artemisinin. Chinese patent NO. CN201711142287.1 discloses a method for purifying artemisinin, which includes the following steps: S1. separating and purifying artemisia annua extract by a column to obtain a column eluate; S2. concentrating the column eluate at 40-85° C., the volume of column eluate after concentration is 1/10- 1/20 of that before concentration. Standing for crystallization, drying the crude product for later use; S3. Adding alcohol to the crude product, dissolving, standing for crystallization, filtering, and taking the crystal to obtain artemisinin. The artemisinin purification method provided by the patent can effectively reduce the content of impurity B and control the content of impurity B to less than 0.2%. However, this purification method is not conducive to large-scale industrial production because the purification method is column separation, and the recovery rate from artemisinin is low.

Chinese patent NO. CN201110114772.4 discloses an environmentally friendly artemisinin extraction process, which includes: (1) Drying the raw materials; (2) Preliminary preparation of artemisinin: extracting the treated raw materials with petroleum ether, separating the supernatant, and eluting the supernatant with a silica gel column to obtain the eluent. The eluent is concentrated until crystals are precipitated, and crystallizing for 15-20 hours in the crystallization tank, filtering to obtain coarse artemisinin crystals; (3) Refining of artemisinin: dissolving the crude artemisinin crystals obtained in step (2) in an alcohol precipitation tank, standing still, taking the supernatant and filtering, concentrating the filtrate, and removing the mother liquor after crystallization for 15-20 hours to obtain a refined artemisinin. This method reduces energy consumption and weakens the influence of thermal decomposition of artemisinin on yield during the extraction process. However, this process takes a long time. For example, the time of each crystallization is more than 15 hours, which seriously affects the efficiency of the experiment. The yield of artemisinin is low because the silica gel column is used in the purification process.

### SUMMARY

The problem to be solved in the present invention is to provide an artemisinin separation and purification process with high yield, high purity, low energy consumption rate and less pollution in the purification process.

In order to solve the above-mentioned problems, the present invention provides a process for separating and purifying artemisinin, comprising the following steps:

S1. Concentrating the artemisinin petroleum ether extract to 1/10- 1/50 of the original volume, and pressure filtering to obtain an artemisinin crude crystal 1 and a mother liquor 1, and concentrating the mother liquor 1 under reduced pressure to obtain an artemisinin extract 1. Wherein, the content of artemisinin in the artemisinin petroleum ether extract is 0.1%-1%;

S2. Adding the artemisinin extract 1 to an alcohol aqueous solution, stirring, dissolving and dispersing same, press filtrating or centrifugal separating, to obtain an alcohol water extract 1 and an artemisinin extract 2;

S3. Subjecting the artemisinin extract 2 to S2 so as to obtain an alcohol water extract 2 and an artemisinin extract 3;

S4. Combining the alcohol water extract 1 and 2, and then cooling, crystallizing and pressure filtering to obtain an artemisinin crude crystal 2 and a mother liquor 2;

S5. Combining the artemisinin crude crystal 1 and the artemisinin crude crystal 2 to obtain a mixture of crude artemisinin crystal, dissolving the mixture of crude artemisinin crystal in an alcohol solvent, filtering, heating the resulting filtrate to 40-70° C. and concentrating under reduced pressure. The volume-to-mass ratio of the concentrated liquid to the crude crystal mixture before concentration is 5-20 L/kg, then the refined artemisinin product is obtained by cooling, filtering and drying. Wherein, the volume-to-mass ratio of the alcohol solvent to the mixture of crude artemisinin crystal is 20-100 L/kg.

Preferably, in S1, the concentration temperature is 50-70° C. Concentrating at this temperature can ensure rapid removal of the solvent without affecting the structure of the active ingredients in the extract. The test results show that when the temperature is higher than 70° C., the yield of artemisinin products is significantly reduced.

Preferably, in S2, the volume fraction of the alcohol aqueous solution is 10-50%, and the alcohol is methanol, ethanol, n-propanol, isopropanol, n-butanol or tert-butanol.

Preferably, in S1, the content of artemisinin in the artemisinin petroleum ether extract is 0.1%-0.5%.

The main components of the extract are artemisinin, arteannuin B, arteannuin C, artemisinic acid and dihydroartemisinic acid.

In a preferred example of the present invention, the artemisinin content in the artemisinin petroleum ether extract in S1 is 0.15%.

Preferably, the preparation process of the artemisinin petroleum ether extract includes the following steps: mixing the artemisia annua leaves with 3-10 times its volume of petroleum ether, heating and extracting at 40-50° C. for 4-8 hours, repeating 2-3 times, and combining the extracts to obtain the artemisinin petroleum ether extract.

Preferably, in S5, the alcohol solvent is methanol, ethanol, n-propanol, isopropanol, n-butanol or tert-butanol.

More preferably, in S5, the alcohol solvent is isopropanol, n-butanol or tert-butanol. When this preferred alcohol solvent is adopted, the amount used when dissolving the crude artemisinin is 20%-50% of other solvents, which can reduce the amount of organic solvents, save production costs, reduce the dissolution loss of artemisinin, also improve the product recovery rate.

Preferably, in S2, the volume mass ratio of the alcohol aqueous solution to the artemisinin extract 1 is (4-6):1.

Preferably, in S5, the way of the cooling is gradient cooling, and the specific operation is as follows: cooling to room temperature naturally, continuing to heat and stir for 40-70 min; then cooling to 15-20° C. within 1-2 h and continuing to heat and stir for 40-70 min; cooling to 0-10° C. within 1-2 h and t continuing to heat and stir for 1.5-3 h.

Gradient cooling can make the artemisinin crystals have a good crystal form, and the cooling time is controlled within 1-2 h, which is conducive to the formation of artemisinin crystals with better purity and better crystal form.

More preferably, in the step S5, the stirring speed during the gradient cooling is 10-30 rad/min. The better artemisinin crystal form is more convenient to be formed in the above-mentioned stirring speed range. The obtained needle crystal form has a larger particle size, also the content and purity of the product is high.

Preferably, in the step S5, the specific operation of the filtering is: sequentially performing filler adsorption filtration and filter bag fine filtration. Wherein, the filler is diatomaceous earth or activated carbon. Washing with petroleum ether during filtration. Some insoluble impurities are removed in the filtering step.

Preferably, in the step S5, a double cone dryer is used for drying, the drying temperature is 40-60° C., and the drying time is 3-6 hours.

More preferably, the temperature of the drying is 50° C., the time of the drying is 3-5 h.

Preferably, the artemisinin extract 3 repeats step S2 at least once to obtain the artemisinin extract and the alcohol water extract N. The alcohol water extract N is used to dissolve the artemisinin extract 1 in the next batch of the step S2.

Preferably, the following operations are performed before pressure filtering in the step S1: stirring the concentrated solution while naturally cooling to 25-35° C., keeping the temperature and continuing to stir for 1-2 h. If there is no stirring during the cooling process, it can ensure that the artemisinin crystallization process will not be too fast, thereby avoiding encapsulation of impurities. The preferred method is to crystallize while stirring, and continue to heat and stir for 1-2 hours after the temperature is naturally lowered to 25-35° C., obtain the artemisinin crude crystals 1 through pressure filtration finally. The method controls the cooling speed and the stirring time, and is beneficial to the formation of artemisinin crystals with good crystal form and uniform quality without impurities.

Preferably, the following operations are carried out before the press filtrating in step S2: adding the artemisinin extract 1 to the alcohol aqueous solution, heating to 25-80° C., stirring and dissolving for 1-2 hours, then cooling to 15-40° C.

Preferably, the specific operation of cooling and crystallizing in step S4 is: cooling to −5 to 10° C., crystallizing for 1-2 hours.

Preferably, after the mother liquor 2 is concentrated, the alcohol solvent is recovered and used in step S5. Recycling alcohol solvents can reduce the solvent loss rate and save production costs.

Compared with the prior art, the artemisinin separation and purification process provided by the present invention has the following beneficial effects.

(1) In the prior art, during industrial production, the extract needs to be separated and purified by column and then concentrated. In this process, the separation and purification of artemisinin is completed by silica gel column layer analysis, which requires a large amount of silica gel, increases the loss amount of silica gel and cost. In addition, column loading and unloading are more complicated in this process, and a large amount of organic solvents (petroleum ether/ethyl acetate) are consumed during elution, so there is a higher safety risk. The present invention does not use column separation, on the one hand, it simplifies the process flow, reduces the amount of silica gel used and saves the cost, on the other hand, reduces the safety risk. The present invention can first recover 50-60% of artemisinin through direct crystallization.

(2) The recovery rate of the fine artemisinin obtained by the process provided by the present invention can reach 75%, the purity can reach more than 98%, the crystal shape is regular, the crystal grain size of needle-like crystals is 0.5-1 cm, and the artemisinin melting range is 151-153° C.

### DESCRIPTION OF THE EMBODIMENTS

The present invention will be further explained, and the examples of the present invention will be described.

In the present invention, the meaning of the yield of refined artemisinin product refers to the weight of refined artemisinin product/theoretical weight of artemisininx100% in the unit weight of artemisia annua leaves, for example, the theoretical content of artemisinin in artemisia annua is 15 kg per ton, 14.5 kg of refined artemisinin product is actually obtained in the separation and purification process of the present invention, and the yield of refined artemisinin product is 14.5/15×100%=96.67%.

The purity of fine artemisinin refers to the percentage of the weight of artemisinin in the fine artemisinin relative to the weight of the refined artemisinin product.

### Example 1

A process for separating and purifying artemisinin, comprising the following steps:

S1. Concentrating the artemisinin petroleum ether extract to 1/40 of the original volume under normal pressure and temperature of 50° C., cooling it down to 25° C. while stirring and continuing to keep the temperature and stirring for 1 hour, then pressing and filtering to obtain an artemisinin crude crystal 1 and a mother liquor 1. Concentrating the mother liquor 1 to dryness under reduced pressure to obtain an artemisinin extract 1, wherein, the content of artemisinin in the artemisinin petroleum ether extract is 0.15%;

S2. Adding the artemisinin extract 1 to an alcohol aqueous solution, heating to 25° C., stirring to disperse for 1 h, then cooling to 15° C., press filtrating to obtain an alcohol water extract 1 and an artemisinin extract 2, wherein the alcohol aqueous solution is methanol solution with a volume fraction of 10%, the mass-volume ratio of the artemisinin extract 1 to the alcohol aqueous solution is 5;

S3. Subjecting the artemisinin extract 2 to S2 so as to obtain an alcohol water extract 2 and an artemisinin extract 3. The artemisinin extract 3 repeats step S2 twice to obtain the artemisinin extract and the alcohol water extract N. The alcohol water extract N is used to dissolve the artemisinin extract 1 in the next batch of the step S2;

S4. Combining the alcohol water extract 1 and 2, stirring and cooling down to −5° C., crystallizing at this temperature for 1 h, pressure filtering to obtain an artemisinin crude crystal 2 and a mother liquor 2;

S5. Combining the artemisinin crude crystal 1 and artemisinin crude crystal 2, mixing them with alcohol solvents, heating to 30° C., stirring to dissolve, cooling to 0° C. in the jacket, and then performing adsorption filtration with filler (diatomaceous earth) and bag fine filtration. Heating the filtrate after the fine filtration to 40° C., concentrating under reduced pressure. The volume-to-mass ratio of the concentrated liquid to the crude crystal mixture before concentration is 5 L/kg. Gradient cooling to 0° C., press filtering, after drying in the double cone dryer for 3 hours at 50° C., the refined artemisinin product is obtained.

Wherein, the alcohol solvent is tert-butanol, and the volume-to-mass ratio of the alcohol solvent to the mixture of crude artemisinin crystal is 20 L/kg.

The gradient cooling process is as follows: cooling to room temperature naturally, continuing to stir for 1 h, then cooling to 15° C. within 1 h and continuing to stir for 1 h, cooling to 0° C. within 1 h and continuing to stir for 2 h. The stirring speed during the gradient cooling is 10 rad/min.

In this example, the yield of the refined artemisinin product was 75%, the purity was 98.7%, and the single impurity was not more than 1%.

### Example 2

A process for separating and purifying artemisinin, which differs from Example 1 in that the alcohol solvent in the step S5 is ethanol.

In this example, the yield of the refined artemisinin product was 69%, the purity was 96.4%, and the single impurity was not more than 1%.

### Example 3

A process for separating and purifying artemisinin, comprising the following steps:

S1. Concentrating the artemisinin petroleum ether extract to 1/50 of the original volume under normal pressure and temperature of 70° C., cooling it down to 25° C. while stirring and continuing to keep the temperature and stirring for 1 hour, then pressing and filtering to obtain an artemisinin crude crystal 1 and a mother liquor 1. Concentrating the mother liquor 1 to dryness under reduced pressure to obtain an artemisinin extract 1, wherein, the content of artemisinin in the artemisinin petroleum ether extract is 0.5%;

S2. Adding the artemisinin extract 1 to an alcohol aqueous solution, heating to 25° C., stirring to disperse for 1 h, then cooling to 40° C., Centrifugal separating to obtain an alcohol water extract 1 and an artemisinin extract 2, wherein the alcohol aqueous solution is tert-butanol solution with a volume fraction of 10%, the mass-volume ratio of the artemisinin extract 1 to the alcohol aqueous solution is 4;

S3. Subjecting the artemisinin extract 2 to S2 so as to obtain an alcohol water extract 2 and an artemisinin extract 3. The artemisinin extract 3 repeats step S2 once to obtain the artemisinin extract and the alcohol water extract N. The alcohol water extract N is used to dissolve the artemisinin extract 1 in the next batch of the step S2;

S4. Combining the alcohol water extract 1 and 2, stirring and cooling down to 10° C., crystallizing at this temperature for 1.5 h, pressure filtering to obtain an artemisinin crude crystal 2 and a mother liquor 2;

S5. Combining the artemisinin crude crystal 1 and artemisinin crude crystal 2, mixing them with alcohol solvents, heating to 50° C., stirring to dissolve, cooling to 0° C. in the jacket, and then performing adsorption filtration with filler (diatomaceous earth) and bag fine filtration. Heating the filtrate after the fine filtration to 60° C., concentrating under reduced pressure. The volume-to-mass ratio of the concentrated liquid to the crude crystal mixture before concentration is 20 L/kg. Gradient cooling to 5° C., press filtering, after drying in the double cone dryer for 3 hours at 50° C., the refined artemisinin product is obtained.

The gradient cooling process is as follows: cooling to room temperature naturally, continuing to stir for 1 h, then cooling to 15° C. within 1 h and continuing to stir for 1 h, cooling to 0° C. within 1 h and t continuing to stir for 2 h. The stirring speed during the gradient cooling is 30 rad/min.

Wherein, the alcohol solvent is isopropanol, and the volume-to-mass ratio of the alcohol solvent to the mixture of crude artemisinin crystal is 50 L/kg.

In this example, the yield of the refined artemisinin product was 74%, the purity was 98.7%, and the single impurity was not more than 1%.

### Example 4

A process for separating and purifying artemisinin, which differs from Example 1 in that the details of the step S5 are as follows: combining the artemisinin crude crystal 1 and artemisinin crude crystal 2, mixing them with alcohol solvents, heating to 30° C., stirring to dissolve, cooling to 0° C. in the jacket, and then performing adsorption filtration with filler (diatomaceous earth) and bag fine filtration. Heating the filtrate after the fine filtration to 40° C., concentrating under reduced pressure. The volume-to-mass ratio of the concentrated liquid to the crude crystal mixture before concentration is 5 L/kg.

Natural cooling to 0° C., press filtering, after drying in the double cone dryer for 3 hours at 50° C., the refined artemisinin product is obtained. Wherein, the alcohol solvent is tert-butanol, and the volume-to-mass ratio of the alcohol solvent to the mixture of crude artemisinin crystal is 20 L/kg.

In this example, the yield of the refined artemisinin product was 65%, the purity was 93%, and the single impurity was not more than 2%.

### Example 5

A process for separating and purifying artemisinin, which differs from Example 1 in that the stirring speed during the gradient cooling in the step S5 is 50 rad/min.

In this example, the yield of the refined artemisinin product was 73%, the purity was 96.6%, and the single impurity was not more than 1%.

### Example 6

A process for separating and purifying artemisinin, which differs from Example 1 in that the gradient cooling process in the step S5 is as follows:

Cooling to room temperature naturally, continuing to stir for 1 h, then cooling to 15° C. within 5 h and continuing to stir for 1 h, cooling to 0° C. within 5 h and t continuing to stir for 2 h.

In this example, the yield of the refined artemisinin product was 71%, the purity was 96%, and the single impurity was not more than 1%.

It will be apparent to those skilled in the art that modifications and variations can be made without departing from the scope and spirit disclosed by the appended claims of the present disclosure, and such modifications and variations all fall in the protection extent of the claims of

1. A process for separating and purifying artemisinin, comprising the following steps:
step S1. concentrating an artemisinin petroleum ether extract to 1/10- 1/50 of the original volume, and pressure filtering to obtain a first artemisinin crude crystal and a first mother liquor, and concentrating the first mother liquor under reduced pressure to obtain a first artemisinin extract, wherein, the content of artemisinin in the artemisinin petroleum ether extract is 0.1%-1%;
step S2. adding the first artemisinin extract to an alcohol aqueous solution, stirring, dissolving and dispersing same, press filtrating or centrifugal separating, to obtain a first alcohol water extract and a second artemisinin extract;
step S3. subjecting the second artemisinin extract to the step S2 so as to obtain a second alcohol water extract and a third artemisinin extract;
step S4. combining the first alcohol water extract and the second alcohol water extract, and then cooling, crystallizing and pressure filtering to obtain a second artemisinin crude crystal and a second mother liquor; and
step S5. combining the first artemisinin crude crystal and the second artemisinin crude crystal to obtain a mixture of crude artemisinin crystal, dissolving the mixture of crude artemisinin crystal in an alcohol solvent, filtering, heating the resulting filtrate to 40-70° C. and concentrating under reduced pressure, the volume-to-mass ratio of the concentrated liquid to the crude crystal mixture before concentration is 5-20 L/kg, then the refined artemisinin product is obtained by cooling, filtering and drying, wherein, the volume-to-mass ratio of the alcohol solvent to the mixture of crude artemisinin crystal is 20-100 L/kg.

2. The process for separating and purifying artemisinin according to claim 1, wherein in the step S2, the volume fraction of the alcohol aqueous solution is 10-50%, and the alcohol is methanol, ethanol, n-propanol, isopropanol, n-butanol or tert-butanol.

3. The process for separating and purifying artemisinin according to claim 1, wherein in the step S1, the content of artemisinin in the artemisinin petroleum ether extract is 0.1%-0.5%.

4. The process for separating and purifying artemisinin according to claim 1, wherein in the step S5, the alcohol solvent is methanol, ethanol, n-propanol, isopropanol, n-butanol or tert-butanol.

5. The process for separating and purifying artemisinin according to claim 1, wherein in the step S2, the volume mass ratio of the alcohol aqueous solution to the first artemisinin extract is 4-6:1.

6. The process for separating and purifying artemisinin according to claim 1, wherein in the step S5, the way of the cooling is a gradient cooling, and the specific operation is as follows:
cooling to room temperature naturally, continuing to heat and stir for 40-70 min; then cooling to 15-20° C. within 1-2 h and continuing to heat and stir for 40-70 min; and then cooling to 0-10° C. within 1-2 h and continuing to heat and stir for 1.5-3 h.

7. The process for separating and purifying artemisinin according to claim 6, wherein the stirring speed during the gradient cooling is 10-30 rad/min.

8. The process for separating and purifying artemisinin according to claim 1, wherein the following operations are performed before pressure filtering in the step S1:
stirring the concentrated solution while naturally cooling to 25-35° C., keeping the temperature and continuing to stir for 1-2 h.

9. The process for separating and purifying artemisinin according to claim 1, wherein the following operations are carried out before the press filtrating in the step S2: adding the first artemisinin extract to the alcohol aqueous solution, heating to 25-80° C., stirring and dissolving for 1-2 hours, then cooling to 15-40° C.

10. The process for separating and purifying artemisinin according to claim 1, wherein the specific operation of cooling and crystallizing in the step S4 is: cooling to −5 to 10° C., crystallizing for 1-2 hours.
